(19) United States
(12) Patent Application Publication
(10) Pub. No.: US 2022/0401416 A1
(43) Pub. Date: Dec. 22, 2022

(54) ANGIOTENSIN RECEPTOR BLOCKERS FOR TREATMENT OF FIBROTIC DISEASE

(71) Applicant: Buderer Drug Company, Avon, OH (US)

(72) Inventors: Matthew J. Buderer, Oak Harbor, OH (US); Jason Genin, Avon Lake, OH (US)

(21) Appl. No.: 17/846,126

(22) Filed: Jun. 22, 2022

Related U.S. Application Data
(60) Provisional application No. 63/213,261, filed on Jun. 22, 2021.

(51) Int. Cl. A61K 31/4178; A61K 9/00; A61P 29/00

(52) U.S. Cl. CPC A61K 31/4178; A61K 9/0014; A61K 9/0019; A61P 29/00; A61K 9/0034

(57) ABSTRACT

A method of treating or preventing fibrotic disease is described. The method includes topical administration of a therapeutically effective amount of an angiotensin receptor blocker in a topical formulation to a subject in need thereof. A method of treating Peyronie's disease by administering a therapeutically effective amount of an angiotensin receptor blocker to a subject in need thereof is also described.

### CROSS-REFERENCE TO RELATED APPLICATIONS

This application claims priority to U.S. Provisional Patent Application No. 63/213,261 filed on Jun. 22, 2021, which is hereby incorporated by reference in its entirety.

### BACKGROUND

Skeletal muscle injuries are a common complaint and account for a large number of patients seen in orthopedic clinics. In addition, there is a high recurrence rate of skeletal muscle strain injuries among athletes. However, the standard treatment for muscle injury remains rest, ice, and administration of anti-inflammatory medications, which have limited effectiveness in treating or preventing post-traumatic muscle fibrosis.

Fibrosis, also known as fibrotic scarring, is a pathological wound healing process that involves the blood pressure-regulating renin-angiotensin system. Fibrosis can occur in many tissues in the body, including the lungs, liver, brain, skeletal muscle, and the heart. Repeated injuries, chronic inflammation, and repair are conditions most likely to lead to fibrosis. Modulation of angiotensin II with angiotensin-converting enzyme inhibitors has been shown to decrease fibrosis. Lim et al., Circulation, 103(6), 789-791 (2001). Experiments have also shown that angiotensin II receptor blockade modulates transforming growth factor-β1, which has been shown to prevent muscle regeneration in models of chronic myopathic disease. Cohn et al., Nat. Med. 13(2), 204-210 (2007). However, there remains a need to identify therapeutic agents that can provide effective treatment and prophylaxis of muscle injury such as fibrotic disease.

### SUMMARY OF THE INVENTION

In one aspect, the present invention provides a method of treating fibrotic disease. The method includes topical administration of a therapeutically effective amount of an angiotensin receptor blocker in a topical formulation to a subject in need thereof. In some embodiments, the angiotensin receptor blocker is selected from the group consisting of azilsartan, candesartan, eprosartan, irbesartan, losartan, olmesartan, telmisartan, and valsartan, while in a further embodiment the angiotensin receptor blocker is losartan. In additional embodiments, the topical formulation is a cream or gel. In yet further embodiments, the fibrotic disease is Peyronie's disease. In further embodiments, the topical formulation is administered to the penis of the subject.

In some embodiments, the method further comprises administration of an additional pharmaceutical agent. For example, in some embodiments the additional pharmaceutical agent is an additional agent for treating fibrotic disease, in further embodiments the additional pharmaceutical agent is a vasodilator, and in yet further embodiments the additional pharmaceutical agent is an antibacterial, antiviral, or antifungal agent.

Another aspect of the present invention provides a method of treating Peyronie's disease. The method includes administering a therapeutically effective amount of an angiotensin receptor blocker to a subject in need thereof. In some embodiments, the angiotensin receptor blocker is selected from the group consisting of azilsartan, candesartan, eprosartan, irbesartan, losartan, olmesartan, telmisartan, and valsartan. In some embodiments, the angiotensin receptor blocker is administered topically or by injection. In further embodiments, the angiotensin receptor blocker is administered by intracavernosal injection. In yet further embodiments, the angiotensin receptor blocker is topically administered to the penis of the subject.

### BRIEF DESCRIPTION OF THE FIGURES

The present invention may be more readily understood by reference to the following drawing, wherein:

FIG. 1 provides a table showing the change in pain reported by patients before and after using a topical composition including losartan.

### DETAILED DESCRIPTION OF THE INVENTION

The present invention provides a method of treating fibrotic disease is described. The method includes topical administration of a therapeutically effective amount of an angiotensin receptor blocker in a topical formulation to a subject in need thereof. The present invention also provides a method of treating Peyronie's disease by administering a therapeutically effective amount of an angiotensin receptor blocker to a subject in need thereof.

### Definitions

The term “treatment,” “treating,” or other equivalents encompasses the amelioration, cure, maintenance (i.e., the prevention of relapse), improvement, and/or reversal of the symptoms of a cardiovascular disease or pathological condition being treated. Treatment after a disease or disorder has started or manifested aims to reduce, ameliorate, or altogether eliminate the disorder, and/or its associated symptoms, to prevent it from becoming worse, or to prevent the disorder from re-occurring once it has been initially eliminated (i.e., to prevent a relapse). In certain embodiments, treatment does not include prevention.

Prevention, as used herein, refers to therapy providing a benefit to a subject having an increased risk of being afflicted with a condition or disease such as fibrotic disease, including avoidance of or a decrease of one or more symptoms of the disease should fibrotic disease occur.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses. An effective amount, on the other hand, is an amount sufficient to provide a significant chemical effect.

As used herein, the term “diagnosis” can encompass determining the likelihood that a subject will develop a disease, or the existence or nature of disease in a subject. The term diagnosis, as used herein also encompasses determining the severity and probable outcome of disease or episode of disease or prospect of recovery, which is generally referred to as prognosis). “Diagnosis” can also encompass diagnosis in the context of rational therapy, in which the diagnosis guides therapy, including initial selection of therapy, modification of therapy (e.g., adjustment of dose or dosage regimen), and the like.

A “subject,” as used herein, can be any animal, and may also be referred to as the patient. Preferably the subject is a vertebrate animal, and more preferably the subject is a mammal, such as a domesticated farm animal (e.g., cow, horse, pig) or pet (e.g., dog, cat). In some embodiments, the subject is a human.

Unless defined otherwise, all technical and scientific terms used herein have the same meaning as commonly understood by one of ordinary skill in the art to which this invention belongs.

### Treatment or Prevention of Fibrotic Disease

In one aspect, the present invention provides a method of treating or preventing fibrotic disease, comprising topical administration of a therapeutically effective amount of an angiotensin receptor blocker in a topical formulation to a subject in need thereof. The term “fibrotic disease” as used herein refers to conditions where the fibroproliferative response produces an abnormal accumulation of fibrocellular scar tissue that compromises the normal architecture and function of the affected tissue. Non-limiting examples of fibrotic disease include Peyronie's disease, myofibrosis, Raynaud's syndrome, psoriasis plaques, eczema, keloid scars, pulmonary fibrosis, liver fibrosis, renal fibrosis, and vascular fibrosis.

While different types of fibrotic disease share many biochemical features, they are diagnosed differently, depending on the specific type of fibrotic disease. For example, pulmonary fibrosis can be diagnosed by spirometry, high-resolution computed tomography, or lung biopsy, while muscle injuries that can lead to fibrotic disease can be diagnosed using a variety of techniques known to those skilled in the art. Jarvinin et al., Am J Sports Med., 33(5):745-64 (2005). Risk factors that cause a subject to have an increased risk of developing fibrotic disease also vary depending on the tissue involved, but are known to those skilled in the art.

In some embodiments, a method of treating or preventing keloid scarring is provided. The method includes topical administration of a therapeutically effective amount of an angiotensin receptor blocker in a topical formulation to a subject having a keloid scar, or being at an increased risk for developing one. A keloid is an enlarged, raised scar that can be pink, red, skin-colored or darker than the surrounding skin, and is caused by excess collagen in the skin during healing.

In a further embodiment, the method of treating or preventing fibrotic disease includes reducing the pain felt by the subject due to fibrotic disease. The inflammation present in fibrotic disease can be a source of significant discomfort, and by reducing inflammation via a reduction of TGF-β, the angiotensin receptor blocker can reduce the pain felt by the subject.

One aspect of the present invention includes topical administration of an angiotensin receptor blocker in a topical formulation to a subject. The term “topical administration” is used in its conventional sense to mean delivery of the angiotensin receptor blocker in a topical formulation to a portion or region of the skin or mucosa of a subject. A variety of topical formulations are described herein, including, for example, cream or gel formulations. In some embodiments, the topical formulation is administered to the penis of the subject.

In some embodiments, topical administration provides transdermal delivery of the angiotensin receptor blocker. The term “transdermal” delivery includes both transdermal (or “percutaneous”) and transmucosal administration, i.e., delivery by passage of a drug through the body surface, i.e., the skin or mucosal tissue. “Transdermal” delivery is also intended to encompass delivery of a drug by passage across scrotal tissue.

The method includes administration of a therapeutically effective amount of an angiotensin receptor blocker. Angiotensin receptor blockers are also known as angiotensin II receptor antagonists. Angiotensin receptor blockers work by blocking angiotensin II from interacting with AT1 receptors. In some embodiments, the angiotensin receptor blocker is selected from the group consisting of azilsartan, candesartan, eprosartan, irbesartan, losartan, olmesartan, telmisartan, and valsartan, while in a further embodiment the angiotensin receptor blocker is losartan. Angiotensin receptor blockers include bi-phenyl tetrazole derivatives, and non-bi-phenyl tetrazole derivatives. Singh K. & Karnik, S., Curr Drug Targets, 21(2):125-131 (2020). A further example of an angiotensin receptor blocker is KT3-671. Takata et al., J Cardiovasc Pharmacol., 37(4):427-36 (2001).

The amount of angiotensin receptor blocker administered to the subject can vary depending on the activity of the specific angiotensin receptor blocker being used. Typically, the composition will include from about 0.1 percent to about 10 percent by weight of the angiotensin receptor blocker. Preferably, the composition includes from about 1 percent to about 5 percent by weight of the angiotensin receptor blocker.

In some embodiments, the invention provides a method for preventing fibrotic disease (e.g., Peyronie's disease). Angiotensin receptor blockers can be administered prophylactically to a subject in advance of the occurrence of fibrotic disease. Prophylactic (i.e., preventive) administration is effective to decrease the likelihood of the subsequent occurrence of fibrotic disease in the subject, or decrease the severity of fibrotic disease that subsequently occurs. A subject in need of prophylactic treatment can be a subject that is at elevated risk of developing fibrotic disease, such as a subject who has suffered an injury or has a genetic predisposition to developing one or more types of fibrotic disease.

Topical administration of an angiotensin receptor blocker can also be used to prevent or decrease the risk that a subject will develop fibrotic disease. In particular, the method can be used to decrease the risk that a subject having an increased risk of developing fibrotic disease will develop fibrotic disease. For example, surgeries such as hysterectomy, plastic surgery, and treatment of retinal detachment can increase the risk that a subject will develop scar tissue (e.g., adhesions) after surgery. Accordingly, topical administration of an angiotensin receptor blocker can be used to prevent or reduce the risk that a subject will develop scar tissue after surgery.

The invention is inclusive of the compounds described herein in any of their pharmaceutically acceptable forms, including isomers (e.g., diastereomers and enantiomers), tautomers, salts, solvates, polymorphs, prodrugs, and the like. In particular, if a compound is optically active, the invention specifically includes each of the compound's enantiomers as well as racemic mixtures of the enantiomers. It should be understood that the term “compound” includes any or all of such forms, whether explicitly stated or not (although at times, “salts” are explicitly stated).

In some embodiments, the angiotensin receptor blocker can be administered together with an additional pharmaceutical agent, including vasodilators (e.g., nitrovasodilators), alpha receptor blocking agents, ergot alkaloids, antihypertensive agents, calcium channel blockers, naturally occurring, semisynthetic and synthetic prostaglandins, and vasoactive intestinal peptides. Examples of suitable vasodilators include be alprostadil (Prostaglandin E₁), papaverine, and phentolamine. The amount of the angiotensin receptor blocker can be great then, equal to, or less than the amount of the additional pharmaceutical agent. For example, the ratio of the angiotensin receptor blocker to the additional pharmaceutical agent can range from about 10:1 to about 1:10, or from about 5:1 to about 1:5, or from about 2:1 to about 1:2.

In some embodiments, the additional pharmacological agents that may be optionally delivered along with the angiotensin receptor blocker includes an analgesic (e.g., NSAIDs such as diclofenac sodium) or anesthetic agent (i.e., substances or compounds that induce insensitivity to pain such as a temporary loss of sensation). Non-limiting examples of acceptable anesthetics that may be so used include lidocaine, tetracaine, proparacaine, procaine or dyclonine.

In some embodiments, the additional pharmaceutical agent is an antibacterial, antiviral, or antifungal agent. Such agents can be included to treat or prevent infection at the site where the composition is being applied. Those having ordinary skill in the art will can select the appropriate agent(s) and their concentration(s).

In some embodiments, the composition includes an antibacterial agent. The choice of antibacterial agent will vary depending on the specific bacteria and the severity of the patient's condition. Examples of antibacterial agents include antibacterial agents, such as quinolones, e.g., ciprofloxacin, ofloxacin, moxifloxacin, methoxyfloxacin, pefloxacin, norfloxacin, sparfloxacin, temafloxacin, levofloxacin, lomefloxacin, and cinoxacin; penicillins, e.g., cloxacillin, benzylpenicillin, and phenylmethoxypenicillin; aminoglycosides, e.g., erythromycin and other macrolides; and antitubercular agents, such as rifampicin and rifapentine.

In some embodiments, an antiviral agent is included in the composition. The choice of antiviral agent will vary depending on the specific virus and the severity of the patient's condition. Examples of antiviral agents include abacavir, acyclovir, adefovir, amantadine, amprenavir, ampligen, arbidol, atazanavir, atripla, balavir, boceprevirertet, cidofovir, combivir, dolutegravir, darunavir, delavirdine, didanosine, docosanol, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon types I-III, lamivudine, lopinavir, loviride, maraviroc, moroxydine, methisazone, nelfinavir, nevirapine, nexavir, oseltamivir (Tamiflu), peginterferon α-2a, penciclovir, peramivir, PF-429242, pleconaril, podophyllotoxin, raltegravir, ribavirin, rimantadine, ritonavir, pyramidine, saquinavir, sofosbuvir, stavudine, tea tree oil, telaprevir, tenofovir, tenofovir disoproxil, tipranavir, trifluridine, trizivir, tromantadine, truvada, traporved, valaciclovir (Valtrex), valganciclovir, vicriviroc, vidarabine, viramidine, zalcitabine, zanamivir (Relenza), and zidovudine.

In some embodiments, an antifungal agent is included in the composition. The choice of antifungal agent will vary depending on the patient's condition and/or the risk of particular fungal infection. Examples of antifungal agents include polyenes such as amphotericin B, candicidin, filipin, hamycin, natamycin, nystatin, and rimocidin; azoles such as bifonazole, butoconazole, clotrimazole, econazole, fenticonazole, isoconazole, ketoconazole, luliconazole, miconazole, omoconazole, oxiconazole, sertaconazole, sulconazole, and ioconazole; and triazoles such as albaconazole, efinaconazole, epoxiconazole, fluconazole, isavuconazole, itraconazole, posaconazole, propiconazole, ravuconazole, terconazole, and voriconazole, as well as additional types of antifungal agents such as thiazoles, allylamines, echinocandins, and triterpenoids.

Topical administration of angiotensin receptor blockers to treat or prevent fibrotic disease can also be carried out together with other methods for treating or preventing fibrotic disease. For example, topical administration of angiotensin receptor blockers can be carried out in conjunction with prolotherapy, also known as non-surgical ligament and tendon reconstruction and regenerative joint injection, for stimulating the body's healing process.

### Treatment or Peyronie's Disease

Another aspect of the present invention provides a method of treating Peyronie's disease. The method includes administering a therapeutically effective amount of an angiotensin receptor blocker to a subject in need thereof. In some embodiments, the angiotensin receptor blocker is selected from the group consisting of azilsartan, candesartan, eprosartan, irbesartan, losartan, olmesartan, telmisartan, and valsartan.

Peyronie's disease (PD; a.k.a. Peyronie's disorder) is a type of fibrotic disease involving the growth of fibrous plaques in the soft tissue of the penis. Specifically, scar tissue forms in the tunica albuginea, the thick sheath of tissue surrounding the corpora cavernosa, causing pain, abnormal curvature, erectile dysfunction, indentation, loss of girth and shortening. Peyronie's disease can be conclusively diagnosed using an ultrasound which can show the presence of scar tissue. However, computed tomography and radiography can also be used to image and diagnose Peyronie's disease. Parmar et al., Curr Opin Urol., 30(3):283-289 (2020).

The method of treating Peyronie' s disease can result in the improvement of one or more symptoms of Peyronie's disease. In some embodiments, the treatment of Peyronie's disease results in reduction of PD plaques. In other embodiments, the treatment results in improvement of erectile dysfunction. In certain embodiments, the erectile dysfunction is associated with PD.

Any method known to those skilled in the art can be used to administer the angiotensin receptor blocker to the subject. For example, the angiotensin receptor blocker can be administered orally, topically, or by injection. In some embodiments, the angiotensin receptor blocker is administered topically or by injection. The pharmaceutical formulation can be administered topically and/or transdermally to the penis of the subject. In some embodiments, the angiotensin receptor blocker is topically administered to at least a portion of the penis of the subject.

In some embodiments, the angiotensin receptor blocker is administered by intracavernosal injection. The term “intracavernosal” as used herein refers to an injection into one or both corpora of the corpora cavernosal tissues of the penis. In certain embodiments of the methods, the pharmaceutical formulation is not administered (e.g., intracavernosally injected) directly into the area of the fibrotic disease. In particular embodiments of the methods for treating Peyronie's disease, the pharmaceutical formulation is not administered (e.g., intracavernosally injected) directly into the area of the Peyronie's disease. In other embodiments of the methods for treating Peyronie's disease, the pharmaceutical formulation is administered (e.g., intracavernosally injected) at the base of the penis about 2 cm from where the penis attaches to the abdomen.

The angiotensin receptor blocker can be administered in a pharmaceutical formulation comprising a sterile liquid composition so that the pharmaceutically acceptable excipient or carrier is suitable for intracavernosal injection. In other embodiments, the pharmaceutically acceptable excipient or carrier is suitable for topical or transdermal administration, and the formulation comprises a composition to be applied to a body surface, such as an ointment, cream, gel or lotion.

### Administration and Formulation

The present invention also provides pharmaceutical compositions that include an angiotensin receptor blocker as an active ingredient, and a pharmaceutically acceptable liquid or solid carrier or carriers, in combination with the active ingredient. Any of the angiotensin receptor blockers described herein can be included in pharmaceutical compositions of the invention.

The phrases “pharmaceutically acceptable” or “pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated.

The active compositions of the present invention may include classic pharmaceutical preparations. Administration of these compositions according to the present invention may be via any common route so long as the target tissue is available via that route. This includes oral, nasal, or buccal. Alternatively, administration may be by intradermal, subcutaneous, intramuscular, intraperitoneal or intravenous injection, drip infusion, transdermal patch, or by direct injection into cardiac tissue. Typical forms of administration include topical and intravenous administration.

Examples of formulations for topical drug delivery include ointments, creams, and gels. Ointments are semisolid preparations which are typically based on petrolatum or other petroleum derivatives. Creams containing the selected active agent include viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. Examples of suitable emulsifiers include surfactants and dimethyl sulfoxide. The specific ointment or cream base to be used, as will be appreciated by those skilled in the art, is one that will provide for optimum drug delivery. As with other carriers or vehicles, an ointment base should be inert, stable, non-irritating and non-sensitizing.

Examples of cream formulations include a first formulation including 2% losartan potassium cream that includes losartan potassium, DMSO, in a vanishing/penetrating cream base, and a second formulation which includes diclofenac sodium, losartan potassium, magnesium sulfate heptahydrate, DMSO, 25% menthol solution, in a vanishing/penetrating cream base.

The angiotensin receptor blocker can also be topically formulated as an aqueous or gel formulation. For example, a topical solution can be used that includes losartan potassium, diclofenac sodium, magnesium chloride hexahydrate, in an alcohol based topical solution, or same solution with a polymer added for a gel formation.

Conventional transdermal drug delivery systems include transdermal “patches” wherein the agent is typically contained within a laminated structure that serves as a drug delivery device to be affixed to the skin. In such a structure, the drug composition is typically contained in a layer, or “reservoir,” underlying an upper backing layer. The laminated device may contain a single reservoir, or it can contain multiple reservoirs. The reservoir can comprise a polymeric matrix of a pharmaceutically acceptable contact adhesive material that serves to affix the system to the skin during drug delivery. Non-limiting examples of suitable skin contact adhesive materials include polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, can be either a polymeric matrix as described above, or a liquid or hydrogel reservoir, or some other form.

In some embodiments, the angiotensin receptor blocker is administered using an implantable pellet. Implantable pellets can provide controlled release of the angiotensin receptor blocker over an extended period of time. Implantable pellets can be made from a variety of biocompatible materials, such as polylactic acid (PLA), polyglycolic acid (PGA), polycaprolactone (PCL) and the copolymer of PLA and PGA, poly (lactic-co-glycolic acid) (PLGA). Such materials have been viewed as attractive candidates because their degradation products are naturally occurring metabolites, i.e., lactic and glycolic acid.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. Prolonged absorption of the injectable compositions can be brought about by the use in the compositions of agents delaying absorption, for example, aluminum monostearate and gelatin.

The compositions of the present invention generally may be formulated in a neutral or salt form. Pharmaceutically acceptable salt refers to the relatively non-toxic, inorganic and organic acid addition salts of the compounds. These salts can be prepared in situ during the final isolation and purification of the compound, or by separately reacting a vasodilator compound with a suitable counterion and isolating the salt thus formed. Representative counterions include sodium, potassium, calcium, magnesium, ammonium, arginine, diethylamine, ethylenediamine, and piperazine salts, and the like. See for example Haynes et al., J. Pharm. Sci., 94, p. 2111-2120 (2005). For example, losartan can be administered as losartan potassium.

Buffers may also be included to provide a suitable pH value for the formulation. Suitable such materials include sodium phosphate and acetate. Sodium chloride or glycerin may be used to render a formulation isotonic with the blood. If desired, the formulation may be filled into the containers under an inert atmosphere such as nitrogen or may contain an anti-oxidant, and are conveniently presented in unit dose or multi-dose form, for example, in a sealed ampoule.

An effective amount, also referred to as a therapeutically effective amount, of a subject agent or pharmaceutical composition thereof, is an amount sufficient to ameliorate at least one symptom associated with the fibrotic disease (e.g., Peyronie's disease). The therapeutically effective amount to be included in pharmaceutical compositions may depend, in each case, upon several factors, e.g., the type, size and condition of the patient to be treated, the intended mode of administration, the capacity of the subject to incorporate the intended dosage form, etc. One of ordinary skill in the art would be able to determine empirically an appropriate therapeutically effective amount.

The administration of the pharmaceutical composition or formulation to a subject can be intravenous, intraarterial, intraperitoneal, intramuscular, subcutaneous, intrapleural, intrathecal, by perfusion through a regional catheter, or by direct intralesional injection. When administering by injection, for example, the administration may be by continuous infusion, or by single or multiple boluses. The dosage may vary depending upon such factors as the patient's age, weight, gender, general medical condition, and previous medical history.

An example has been included to more clearly describe particular embodiments of the invention. However, there are a wide variety of other embodiments within the scope of the present invention, which should not be limited to the particular example provided herein.

### EXAMPLE

### Example 1: Call-back Program for Compounded Pain Management with Losartan

### Purpose

### Call-Back Program

Buderer Drug Company implemented a call-back program in October 2015 for patients who receive a compounded prescription for pain management. The purpose of the program is to evaluate how well the patient's therapy works and to improve the patient's condition by ensuring the medication is working and refilled or adjusted as needed.

### Losartan Subgroup

The specific purpose of this quality improvement study is to understand the pain relief and satisfaction of a subset of patients who received topical formulations containing losartan, an angiotensin receptor blocker. The purpose of the addition of losartan to the topical formulations is to decrease the production of transforming growth factor beta (TGF-β) as an adjunct for the treatment of controlling fibrosis formation, myopathies, pain and inflammation.

### Methods

### Call-Back Program

Patients receiving a compounded prescription for pain are contacted on the telephone by a Buderer Drug staff member (or pharmacy student) about 1 week before a refill is needed. The length of time between the initial prescription and the follow-up phone call varies based on staff availability and ability to reach the patient. Using a standardized script, the staff member administers an eight-item survey with the patient. Patients may request a refill at this time as well as request a call-back from a pharmacist to ask questions or discuss medication adjustments.

### Losartan Subgroup

For this particular study, follow-up calls were made up to 1.5 years after the initial prescription was received.

### Results of Losartan Subgroup

Included in this report is a subset of patients who received at least one prescription of a losartan containing topical between November 2020 and first week of June 2022. In April and June 2022, calls were made to all 58 of these patients; 53 (91%) responded.

Patients were prescribed the medication most commonly for inflammatory conditions including tendonitis and arthritis as well as scarring. The medication was delivered in a cream or gel base for 41 (77%) patients and 12 (23%) had a liquid formulation. A majority of the patients reported using the medication 2 to 4 times daily.

### Pain

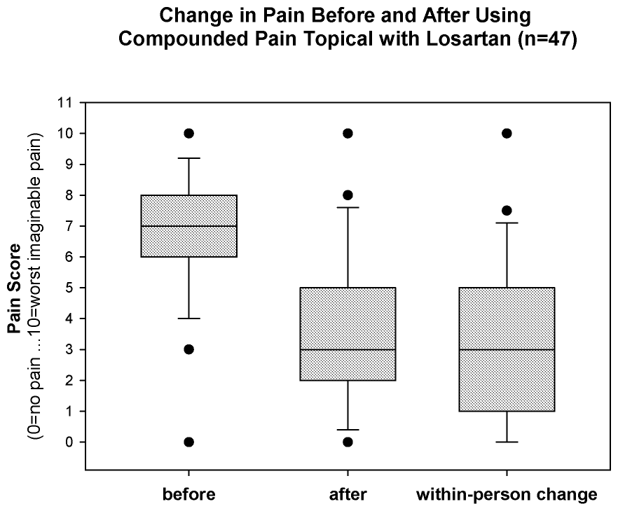

Patients were asked to rate their pain on a 10-point scale from 0 (no pain) to 10 (worst imaginable pain) using 2 questions: “How would you rate your pain before you started taking this medicine?”, and “How would you rate your pain now after you have been taking this medicine?”. The within-person change in pain was calculated as the patient's ‘before’ pain score minus their ‘after’ pain score. Pain dropped from a median of 7 (before medication) to 3 (after medication), with a median within-patient decrease of 3 points (interquartile range 1 to 5). The results are shown in FIG. 1.

### Refills

Overall, 24% of the patients who received the losartan containing medication refilled their prescription at least once.

### Conclusion

Patients who received individualized compounded pain medication containing losartan for inflammatory conditions reported a reduction in pain.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of treating or preventing fibrotic disease, comprising topical administration of a therapeutically effective amount of an angiotensin receptor blocker in a topical formulation to a subject in need thereof.

2. The method of claim 1, wherein the angiotensin receptor blocker is selected from the group consisting of azilsartan, candesartan, eprosartan, irbesartan, losartan, olmesartan, telmisartan, and valsartan.

3. The method of claim 1, wherein the angiotensin receptor blocker is losartan.

4. The method of claim 1, wherein the method comprises treatment of fibrotic disease.

5. The method of claim 1, wherein the method comprises prevention of fibrotic disease.

6. The method of claim 1, wherein the topical formulation is a cream or gel.

7. The method of claim 1, wherein the fibrotic disease is Peyronie's disease.

8. The method of claim 1, wherein the topical formulation is administered to the penis of the subject.

9. The method of claim 1, wherein the subject is human.

10. The method of claim 1, wherein the method further comprises administration of an additional pharmaceutical agent.

11. The method of claim 10, wherein the additional pharmaceutical agent is an additional agent for treating fibrotic disease.

12. The method of claim 11, wherein the additional pharmaceutical agent is a vasodilator.

13. The method of claim 10, wherein the additional pharmaceutical agent is an antibacterial, antiviral, or antifungal agent.

14. The method of claim 10, wherein the additional pharmaceutical agent is an analgesic or anesthetic agent.

15. The method of claim 1, wherein treating or preventing fibrotic disease comprises reducing the pain felt by the subject due to fibrotic disease.

16. A method of treating Peyronie's disease, comprising administering a therapeutically effective amount of an angiotensin receptor blocker to a subject in need thereof.

17. The method of claim 16, wherein the angiotensin receptor blocker is selected from the group consisting of azilsartan, candesartan, eprosartan, irbesartan, losartan, olmesartan, telmisartan, and valsartan.

18. The method of claim 16, wherein the angiotensin receptor blocker is administered topically or by injection.

19. The method of claim 18, wherein the angiotensin receptor blocker is administered by intracavernosal injection.

20. The method of claim 18, wherein the angiotensin receptor blocker is topically administered to at least a portion of the penis of the subject.
